(19) United States
(12) Patent Application Publication
(10) Pub. No.: US 2020/0306088 A1
(43) Pub. Date: Oct. 1, 2020

(54) GOGGLE STRAP RETENTION SYSTEMS AND METHODS

(71) Applicant: 100% SPEEDLAB, LLC, San Diego, CA (US)

(72) Inventor: Michael D. Young, San Diego, CA (US)

(21) Appl. No.: 16/824,525

(22) Filed: Mar. 19, 2020

Related U.S. Application Data
(60) Provisional application No. 62/826,889, filed on Mar. 29, 2019.

(51) Int. Cl. A61F 9/02

(52) U.S. Cl. CPC A61F 9/027

(57) ABSTRACT

A goggle is described herein. The goggle can include a cage configured to receive an elastic band and be disposed within an opening of the goggle frame. The cage can be made from a stronger material than the goggle frame itself and, thus, allow for a stronger connection of the elastic band to the goggle frame than traditional techniques. The cage can also include features to more securely hold the elastic band within the cage and the cage within the opening of the goggle frame.

### CROSS REFERENCE TO RELATED APPLICATIONS

This application claims the benefit of and priority to U.S. Provisional Patent Application No. 62/826,889 filed Mar. 29, 2019 and entitled “GOGGLE STRAP RETENTION SYSTEMS AND METHODS,” which is incorporated herein by reference in its entirety.

### TECHNICAL FIELD

One or more embodiments relate generally to goggles and, more particularly, to systems and techniques for coupling a goggle strap to a goggle frame.

### BACKGROUND

Sport goggles are worn by users for various sports or activities, such as motorsports, powersports, snowsports, watersports, biking, or the like, to protect wearers' eyes. A sport goggle may be held to the wearer's eyes through a strap. During activities, the goggle may receive forces from, for example, impacts that may stretch or otherwise impart a pulling force on the strap. Additionally, a wearer may remove the goggle by pulling on a frame of the goggle and, thus, stretch the strap. Accordingly, the strap must be secured to the goggle frame in a robust manner to avoid failure of the strap, frame, and/or mechanisms for coupling the strap and frame to each other when the strap is stretched.

Furthermore, sport goggles may be bulky. A bulky goggle may be heavy, may result in additional unwanted impacts, may be harder to wear, may reduce the field of vision of the wearer, and/or may be more difficult to handle in other ways. Thus, there is a need for improved sport goggles.

### SUMMARY

Systems and methods are provided in accordance with one or more embodiments directed to a goggle. In a certain embodiment, a goggle may include a goggle frame, a first outrigger, a second outrigger, an elastic band, a first cage, and a second cage. The goggle frame may include a first end and a second end. The first outrigger may be disposed on the first end of the goggle frame. The second outrigger may be disposed on the second end of the goggle frame and each of the first and second outriggers may include an outrigger opening. The elastic band may include a first end and a second end. The first cage may be disposed within the first outrigger opening. The second cage may be disposed within the second outrigger opening. Each of the first and second cages may include a band opening and at least one protrusion disposed within the band opening. The band opening of the first cage may receive the first end of the elastic band. The band opening of the second cage may receive the second end of the elastic band. The at least one protrusion of each of the first cage and the second cage may be coupled to the elastic band to hold the elastic band within the respective band opening.

In a certain embodiment, each of the first cage and the second cage may further include a first cage fastener opening, the elastic band may include a first band fastener opening, and the goggle may further include a first fastener configured to be disposed through both the first cage fastener opening and the first band fastener opening. In a certain such embodiment, each of the first cage and the second cage may further include a second cage fastener opening, the elastic band may further include a second band fastener opening, the goggle may further include a second fastener configured to be disposed through both the second cage fastener opening and the second band fastener opening, and the first cage fastener opening and the second cage fastener opening may be disposed on opposing sides of the cage.

In a certain embodiment, each of the first cage and the second cage may further include a detent, and each of the first outrigger and the second outrigger may further include a depression disposed within each of the respective outrigger opening and receiving the detent to hold the respective cage within the respective outrigger opening.

In a certain embodiment, the goggle may further include a goggle lens disposed within the goggle frame. The first cage and/or the second cage may be a metal cage. At least one of the protrusions may be a substantially triangular shaped teeth. The elastic band opening may include a substantially rectangular cross section. At least one of the protrusions may extend into the substantially rectangular cross section.

In a certain embodiment, a method of manufacturing the goggle may include forming the goggle frame, forming the first cage, forming the second cage, disposing the first cage within the first outrigger opening, and disposing the second cage within the second outrigger opening. In a certain such embodiment, the first outrigger and the second outrigger may be formed with the goggle frame. In a certain additional such embodiment, the method may further include forming the first outrigger, forming the second outrigger, and coupling the first outrigger and the second outrigger to the goggle frame. In a certain additional such embodiment, the forming of the first cage may include disposing the first end of the elastic band within the band opening of the first cage, and the forming of the second cage may include disposing the second end of the elastic band within the band opening of the second cage.

In a certain embodiment, a method of using the goggle may include receiving a force on the elastic band, stretching the elastic band due to the force, and holding the first end and the second end of the elastic band within the respective band openings.

In another embodiment, a cage may be disposed. The cage may be configured to be disposed within a portion of a goggle frame. The cage may include a cage body configured to be disposed within an opening of a goggle frame, a band opening disposed within the cage body and configured to receive an elastic band, and a protrusion disposed within the band opening and configured to couple to the elastic band to hold the elastic band within the band opening.

In a certain embodiment, the cage may further include a first cage fastener opening configured to receive a first fastener. In a certain such embodiment, the cage may further include a second cage fastener opening configured to receive a second fastener, where the first cage fastener opening and the second cage fastener opening may be disposed on opposing sides of the cage. In a certain additional such embodiment, the cage body may include a detent configured to be disposed within a depression of the goggle frame. In a certain additional such embodiment, the cage body may be metal, the protrusion may be a substantially triangular shaped tooth, the band opening may include a substantially rectangular cross section, and the protrusion may protrude into the substantially rectangular cross section. In a certain additional such embodiment, the cage body may include a first portion and a second portion coupled to the first portion.

In a certain embodiment, a method of manufacturing the cage may include forming the cage body and forming the protrusion. In a certain such embodiment, the cage body and the protrusion may be formed by machining a metal stock. In a certain additional such embodiment, the forming of the cage body may include forming the cage body from sheetmetal to define the band opening and forming the protrusion may include bending the protrusion into the band opening. In a certain additional such embodiment, the cage body and the protrusion may be formed by three-dimensional printing.

The scope of the invention is defined by the claims, which are incorporated into this Summary by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a perspective view of a goggle, in accordance with an embodiment of the invention.

FIG. 2 shows an exploded view of portions of the goggle of FIG. 1, in accordance with an embodiment of the invention.

FIG. 3 shows a perspective view of a portion of the goggle of FIG. 1, in accordance with an embodiment of the invention.

FIGS. 4 and 5 show views of a strap and cage configured to be coupled to a goggle frame, in accordance with embodiments of the invention.

FIG. 6 shows a top view of the goggle of FIG. 1, in accordance with an embodiment of the invention.

FIGS. 7 and 8 show cutaway views of the goggle of FIG. 6, in accordance with embodiments of the invention.

FIG. 9 shows a perspective view of a cage, in accordance with an embodiment of the invention.

FIG. 10 shows a flowchart detailing a method of making the goggle of FIG. 1, in accordance with an embodiment of the invention.

FIG. 11 shows a flowchart detailing a method of making and using the goggle of FIG. 1, in accordance with an embodiment of the invention.

Embodiments of the invention and their advantages are best understood by referring to the detailed description that follows. It should be appreciated that like reference numerals are used to identify like elements illustrated in one or more of the Figures.

### DETAILED DESCRIPTION

A goggle that includes a cage to couple the strap of the goggle to the goggle frame is described herein. The systems and techniques described herein may allow a strap to be coupled to a goggle frame through a cage that offers a smaller form factor and a stronger connection. Thus, the cage can allow for improved strap to goggle frame coupling.

Typically, the strap of a sports goggle is directly coupled to the goggle frame. That is, the goggle frame, which may be a plastic or other type of molded frame, has attachment points on the periphery of the goggle frame configured to receive the strap. As the goggle frame is made of plastic, such attachment points may need to be reinforced, leading to a bulkier goggle frame. Especially as the attachment points are typically on the sides of the goggle frame, the wearer's peripheral vision may be impacted.

A goggle is disclosed in accordance with various embodiments. In a certain embodiment, the goggle may include a goggle frame, a goggle lens, a cage, and an elastic band. The goggle frame may include an outrigger disposed on an end of the goggle frame. The goggle lens may be disposed within the goggle frame. The outrigger may include an outrigger opening that is configured to receive the cage. The cage may include a first opening configured to receive an elastic band and a protrusion disposed within the first opening and configured to couple to the elastic band to hold the elastic band within the first opening.

The cage may be, in certain embodiments, made from metal or another material stronger than the material of the goggle frame. Thus, the cage may be configured to receive a greater force component than typical goggle frame based attachment techniques before failure of the coupling. Additionally, the cage may be made to be less bulky than that of a traditional goggle frame attachment technique. Features within the opening of the cage where the strap is disposed within may aid in the cage holding the elastic band. Features on the outside of the cage may aid in attaching the cage to the opening of the goggle frame and/or outrigger.

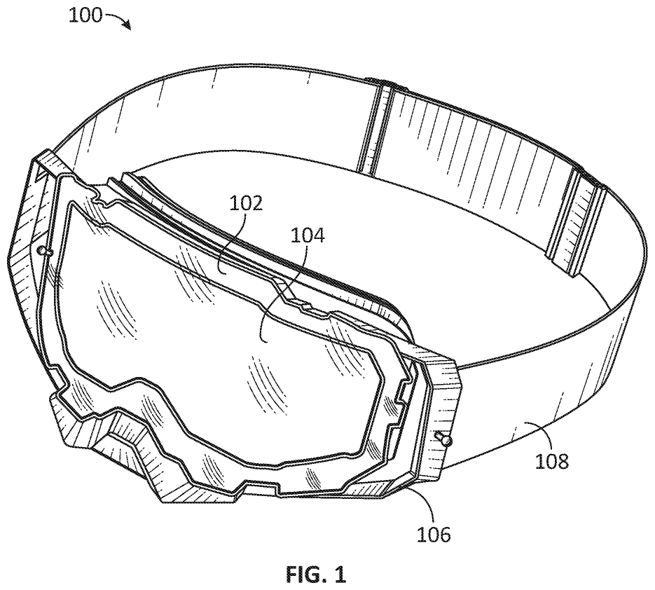

FIG. 1 shows a perspective view of a goggle, in accordance with an embodiment of the invention. FIG. 1 illustrates goggle 100 that includes goggle frame 102, goggle lens 104, outrigger 106, and elastic band 108.

The goggle frame 102 may be configured to receive the goggle lens 104. The goggle frame 102 may be made of any appropriate material, such as one or more of plastics, composites, metals, or other materials. The goggle frame 102 may be formed by, for example, molding, casting, machining, and/or coupling together (e.g., gluing or mechanically fastening) of one or more multiple components.

The goggle frame 102 may include the outrigger 106. The outrigger 106 may, in certain embodiments, be an extension of the goggle frame 102. In other embodiments, the outrigger 106 may be separate from the goggle frame 102. Thus, for example, the outrigger 106 may be formed separate from the goggle frame 102 and coupled to the goggle frame 102 (e.g., mechanically, adhesively, or through another technique). In goggles with outriggers, the outriggers may be disposed on lateral ends of the goggle frame. Thus, for example, a first outrigger may be disposed on a first end of the goggle frame 102 (e.g., the left side of the goggle frame 102) and a second outrigger may be disposed on a second end of the goggle frame 102 (e.g., the right side of the goggle frame 102). Each outrigger may include an opening that receives a corresponding cage.

In certain embodiments, the outrigger 106 may thus define at least a portion of an opening of the goggle frame 102 configured to receive the goggle lens 104. In certain embodiments, the goggle frame 102 may not include an outrigger 106. In such embodiments, the cage described herein may be configured to be disposed within one or more openings of the goggle frame 102 itself. As such, the systems and techniques described herein is applicable to goggles that do not include outriggers as well as goggles that include outriggers.

Elastic band 108 may be coupled to the goggle frame 102. The elastic band 108 may be, for example, a strap that is configured to, when the goggle 100 is worn by a wearer, hold the goggle 100 to the face of the wearer. As such, the elastic band 108 may be configured to be stretched to conform with the wearer's head, to allow for the goggle 100 to be disposed on the wearer's head, to allow for the goggle 100 to be removed from the wearer's head, and/or for other reasons. The elastic band 108 may be adjustable in length to accommodate a variety of head shapes. Additionally, as described herein, the elastic band 108 may be coupled to a cage, and the cage may then accordingly be coupled to the goggle frame 102 and/or the outrigger 106.

In certain embodiments, the goggle frame 102 may include an opening configured to receive the goggle lens 104. In certain such embodiments, the goggle lens 104 may be configured to be disposed within and to decouple from the goggle frame 102 (e.g., to allow for replacement of the goggle lens 104). The goggle lens 104 may be any appropriate goggle lens that allows a wearer to view objects on the other side of the goggle lens 104. The goggle lens 104 may, thus, be an opaque lens configured to allow viewing through the lens by the wearer while protecting the wearer's eyes.

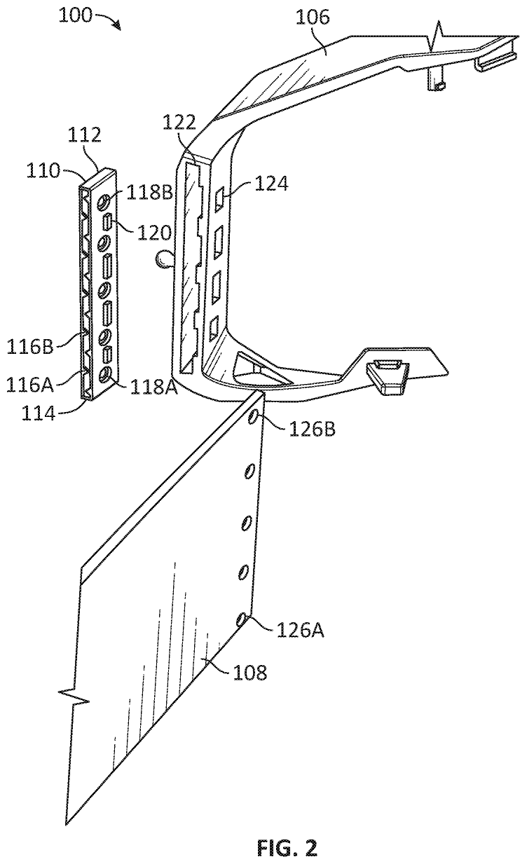

FIG. 2 shows an exploded view of portions of the goggle of FIG. 1, in accordance with an embodiment of the invention. FIG. 2 illustrates an exploded view of the goggle 100 of FIG. 1 that includes the outrigger 106, the elastic band 108, and a cage 110. In certain embodiments, the outrigger 106 may be a portion of the goggle frame 102.

As shown in FIG. 2, the elastic band 108 may be coupled to the cage 110. The cage may then be disposed within an opening 122 of the outrigger 106. Thus, the elastic band 108 may be coupled to the outrigger 106 and, accordingly, the goggle frame, through the cage 110. Such a configuration may allow for the elastic band 108 to have a stronger and more compact coupling to the outrigger 106 and/or the goggle frame 102.

The cage 110 may be, in certain embodiments, made from metal or another material stronger than the material of the goggle frame 102 and/or the outrigger 106 (e.g., a modulus of elasticity of the material of cage 110 may be higher than the modulus of elasticity of the material of the goggle frame 102 and/or the outrigger 106). Thus, coupling the elastic band 108 to the cage 110 and then disposing the cage 110 within the opening 114 may lead to a stronger connection than typical techniques that attach the elastic band directly to the goggle frame and/or the outrigger.

Furthermore, the cage 110 may be more compact (e.g., narrower) than a typical connection while maintaining or improving strength. Thus, the outrigger 106 and/or the goggle frame 102 may be of a smaller size. As, typically, the elastic band 108 is coupled to the sides of the goggle 100 (e.g., disposed to the left and right of the face of the wearer), such a configuration may lead to a more compact goggle frame 102 and/or more compact outrigger 106 that can improve the wearer's field of vision.

The cage 110 may include features for coupling to the elastic band 108 and/or coupling to the outrigger 106. For example, the cage 110 may include a cage body 112. The cage body 112 may include an opening 114. The opening 114 may be substantially rectangular in cross section to receive the elastic band 108. The opening 114 may be of a height and width configured to accommodate the elastic band 108. Thus, the width of the opening 114 may be wider than the thickness of the elastic band 108. The width of the opening 114 may also accordingly be wider than the width of the elastic band 108.

The cage 110 may include features to hold the elastic band 108 when the elastic band 108 is disposed within the opening 114. For example, the cage 110 may include protrusions 116 that aid in holding the elastic band 108 within the opening 114. The protrusions 116 may be, for example, teeth with triangular, square, or other geometries, depressions, adhesives, rods, and/or other types of protrusions or features that may aid in retaining the elastic band 108 within the opening 114. The protrusions 116 may be disposed within a portion of the opening 114. While the embodiment shown in FIG. 2 includes multiple triangular teeth shaped protrusions 116A-B and others, other embodiments may include protrusions and/or retaining features of other geometries and/or may include more or fewer protrusions.

The elastic band 108 may additionally include openings 126A, 126B, and others. Various embodiments may include any number of such openings. The cage may include corresponding openings 118A, 118B, and others. The openings 118A and 118B may be configured to correspond to the openings 126A and 126B so that, when the elastic band 108 is disposed within the opening 114, the opening 118A is aligned with the opening 126A, the opening 118B is aligned with the opening 126B, and so on. When the openings 126A and 126B are aligned with the openings 118A and 118B, respectively, fasteners may be disposed through each pair of openings to aid in holding the elastic band 108 within the opening 114 of the cage 110.

The cage 110 may additionally include detent 120 and other features disposed on an exterior portion of the cage 110. Such features may be configured to interface with corresponding feature 124 within the opening 122. The detent 120 may be, for example, one or more raised surfaces (e.g., protrusion) or depressions, fasteners, adhesives, or other features so that, when the elastic band 108 is pulled, the detent 120 and the corresponding feature 124 may form a mechanical interface (e.g., may include contacting walls) to hold the cage 110 within the opening 122. The corresponding feature 124 within the opening 122 may include depressions to correspond to raised surfaces, raised surfaces to correspond to depressions, and/or corresponding fasteners or features. Thus, the elastic band 108 may be more securely disposed within the opening 114.

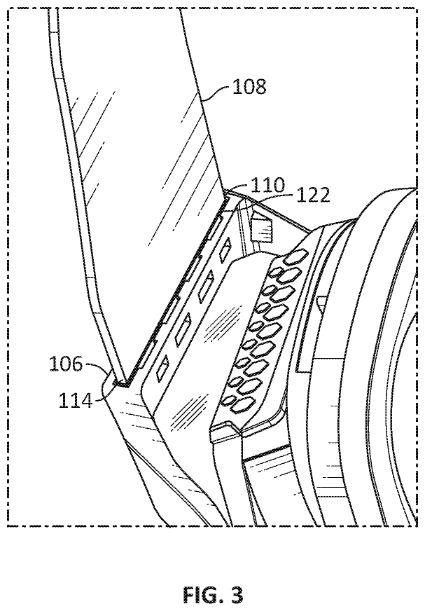

FIG. 3 shows a perspective view of a portion of the goggle of FIG. 1, in accordance with an embodiment of the invention. FIG. 3 illustrates the cage 110 disposed within the opening 122 of the outrigger 106. As such, the cage 110 is coupled to the elastic band 108 and is disposed within the opening 122. The cage 110 may be held within the opening 122 through the features 124 as described herein. The elastic band 108 may also accordingly be held within the opening 114.

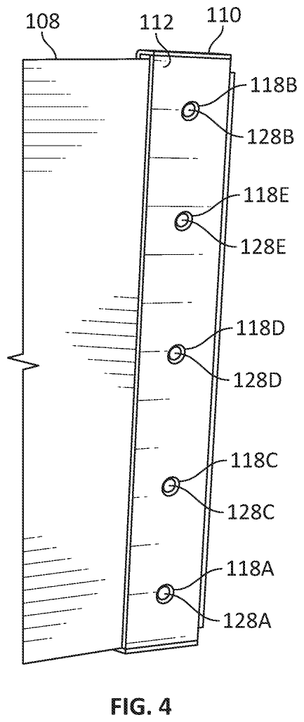

FIGS. 4 and 5 show views of a strap and cage configured to be coupled to a goggle frame, in accordance with embodiments of the invention. FIGS. 4 and 5 show the elastic band 108 coupled to the cage 110. As shown, the cage 110 includes openings 118A-E. The elastic band 108 may include corresponding openings (e.g., openings 126A-E; not shown in FIGS. 4 and 5, but shown in FIG. 2). The openings 118A-E may be aligned with the openings 126A-E and fasteners 128A-E may be disposed through the openings 118A-E and 126A-E. As such, the fasteners 128A-E may aid in holding the elastic band 108 within the opening 114 of the cage 110.

In certain embodiments, the fasteners 128A-E may be, for example, a rivet, a mushroom cap, a bolt and/or a nut, a snap, a protrusion of the cage 110, a dowel, adhesive, teeth, and/or other type of feature configured to further secure the elastic band 108 within the opening 114 of the cage 110. In certain such embodiments, the openings 118A-E may be substantially (e.g., +/−20% in variance) evenly spaced on the cage 110. Thus, the distance between each of the openings 118A-E may be substantially similar. Furthermore, at least two of the openings 118A-E (e.g., the openings 118A and 118B) may be disposed on opposing sides of the cage 110. Disposing the openings in such a manner allows for more even distribution of stresses (e.g., resulting from pulling on the elastic band 108) within portions of the elastic band 108 and/or the cage 110, further increasing strength.

In FIG. 5 may illustrate another embodiment of the cage 110. FIG. 5 may illustrate detents 120A-E on an exterior surface of the cage 110. The detents 120A-E may be, for example, one or more raised surfaces (e.g., protrusion) or depressions, fasteners, adhesives, or other features configured to interface with corresponding features the opening 122 of the outrigger 106 and/or goggle frame 102. Such corresponding features may include depressions to correspond to raised surfaces, raised surfaces to correspond to depressions, and/or corresponding fasteners or other features. Detents 120A-E may allow for the cage 110 to be more securely disposed within the opening 114 and, thus, increase the strength of the coupling of the elastic band 108 to the goggle frame 102 and/or the outrigger 106.

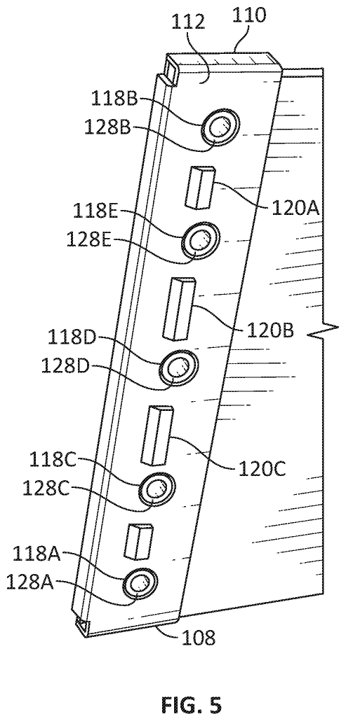

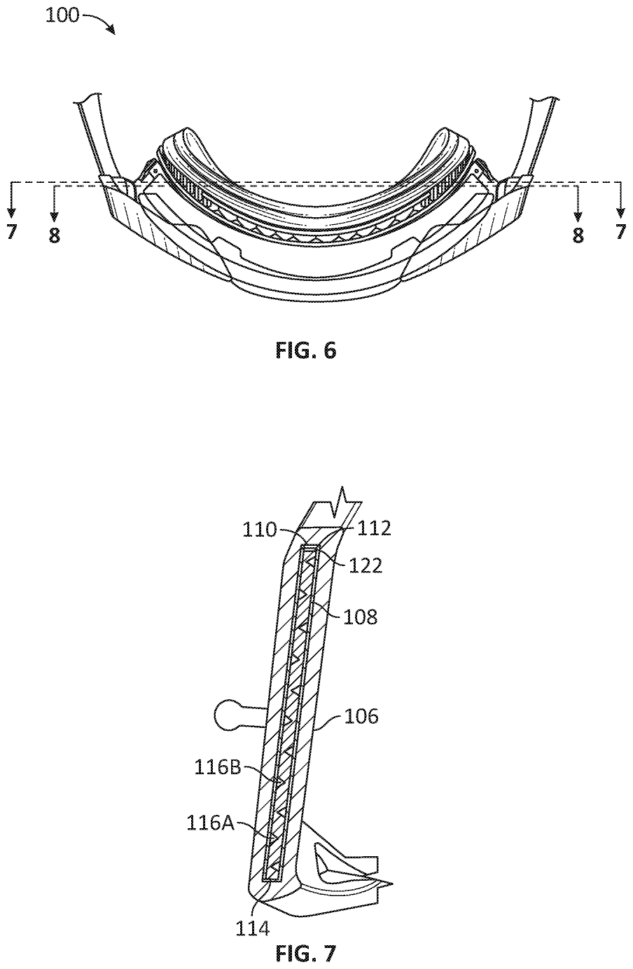

FIG. 6 shows a top view of the goggle of FIG. 1, in accordance with an embodiment of the invention. FIG. 6 is a top view of the goggle 100. FIG. 6 shows planes 7-7 and 8-8. FIGS. 7 and 8 show cutaway views of the goggle of FIG. 6, in accordance with embodiments of the invention. The cutaway view of FIG. 7 is taken along plane 7-7. The cutaway view of FIG. 8 is taken along plane 8-8.

The cutaway view of FIG. 7 illustrates the cage 110 disposed within the opening 122 of the outrigger 106. As shown, the cage 110 may be substantially rectangular in cross-sectional shape and the opening 122 may include a shape that corresponds to the cross-sectional shape of the cage 110 to hold the cage 110 within the opening 122. The cutaway view of FIG. 7 further illustrates the elastic band 108 disposed within the opening 114 of the cage 110. While disposed within the opening 114, the protrusions 116A and 116B, as well as other protrusions illustrated, may grip the elastic band 108 to aid in holding the elastic band 108 within the opening 114. The protrusions may, for example, dig into the side of the elastic band 108 to grip the elastic band 108.

The cutaway view of FIG. 8 illustrates that the detents 120A-D of the cage 110 are disposed within the features 124A-D. While certain embodiments of the features 124A-D may be depressions within the goggle frame 102 and/or the outrigger 106, the embodiment shown in FIG. 8 illustrates that the features 124A-D may also be openings within the goggle frame 102 and/or the outrigger 106. The detents 120A-D may be disposed within at least a portion of the features 124A-D, respectively. The features 124A-D may include one or more surfaces to prevent the detents 120A-D (and, thus, the cage 110) from moving in one or more directions while disposed within the features 124A-D. Certain embodiments may constrain different directions of movement. Such directions may include a direction that prevents rearward movement of the cage 110 due to, for example, a pulling force exerted on the elastic band 108.

Additionally, as shown in FIG. 8, the elastic band 108 may be coupled to the cage 110 through the fasteners 128A-E. Portions of the fasteners 128A-E may be disposed within the openings 118A-E of the cage 110 and openings 126A-E of the elastic band 108 to couple the elastic band 108 to the cage 110.

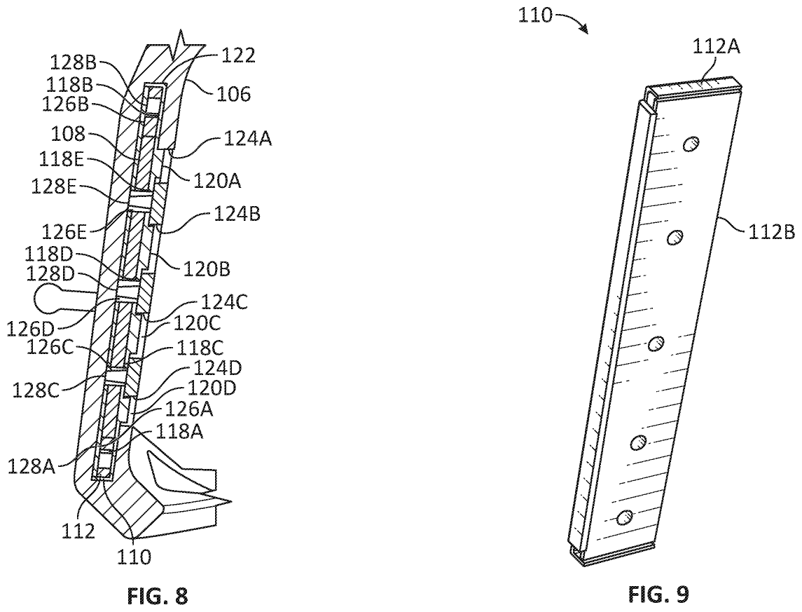

FIG. 9 shows a perspective view of a cage, in accordance with an embodiment of the invention. FIG. 9 illustrates an embodiment of the cage 110. As shown in FIG. 9, the cage 110 may be made of a plurality of separate portions. That is, cage 110 may include cage bodies 112A and 112B. Each of cage bodies 112A and 112B may be, for example, a part manufactured from folded sheetmetal, stamped sheetmetal, machined stock, cast metal, molded composites, or other techniques. In certain embodiments, the cage bodies 112A and 112B may be made from separate techniques.

The separate cage bodies 112A and 112B may then be coupled together (e.g., friction fit, mechanically coupled, adhesively coupled, or coupled through other techniques) to form the cage 110. For example, in certain embodiments, the elastic band 108 may be disposed between the cage bodies 112A and 112B and the cage bodies 112A and 112B may then be accordingly coupled together to form the cage 110. The cage 110 may then be disposed within the opening 122.

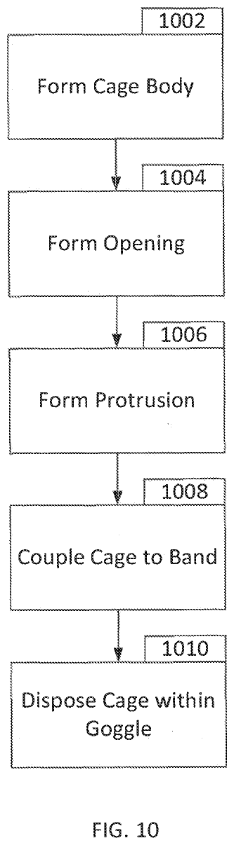

FIG. 10 shows a flowchart detailing a method of making the goggle of FIG. 1, in accordance with an embodiment of the invention. In block 1002, the cage body of the cage may be formed. The cage body may, in certain embodiments, be a metal cage body. The cage body may be formed from bent sheetmetal (e.g., through one or more bending operations performed on a flat pattern), machined from stock, cast, molded, extruded, or otherwise appropriately formed.

After the cage body has been formed in block 1002, the opening may be formed in block 1004. In certain embodiments, the opening may be formed when the cage body is formed. Thus, for example, in embodiments where the body is formed through bent sheetmetal, bending of the sheetmetal may accordingly form the opening. Furthermore, if the cage body is cast, the casting operation may form the opening at the same time as the cage body. In other embodiments, such as embodiments where the cage is machined or cast, the opening may be formed in a separate operation. Thus, for example, the opening may be machined into the cage body.

After the opening has been formed, any protrusions within the opening may be formed in block 1006. For example, in embodiments where the opening and/or the cage body is formed from bent sheetmetal, additional portions of the flat pattern may be bent to form the protrusions. Alternatively or additionally, the protrusions may be machined when the opening is machined. In other embodiments, the protrusions may be formed as a separate component and may be disposed within the opening and coupled to the cage body (e.g., through fasteners, adhesives, welding, or other techniques).

In block 1008, the elastic band may be disposed within the opening to couple the elastic band to the cage. In certain embodiments, the elastic band may be inserted into the opening and held by the protrusions. In other embodiments, the elastic band may first be inserted onto a flat pattern or a partially formed opening. The opening may then be formed around the elastic band. Thus, for example, the elastic band may be disposed within a partially formed opening and the opening may then be finished around the elastic band by, for example, performing the remaining bending steps to form the opening. In such an example, the protrusions may be formed (e.g., bend) before the opening is fully formed. Thus, when the opening is fully bent, the protrusions may accordingly grasp the elastic band in a manner to retain the elastic band.

In block 1010, the cage may be disposed within the goggle frame and/or the outrigger. The cage may, thus, be inserted into an opening of the goggle frame and/or the outrigger. The cage may be held within the opening through features on the exterior of the cage and/or the opening of the goggle frame and/or the outrigger. In certain embodiments, the cage may be held within the opening by adhesives or other coatings. Thus, for example, the opening and/or the cage may include a compressible coating that is configured to be deformed when the cage is disposed within the opening, further aiding in preventing the cage from separating from the opening.

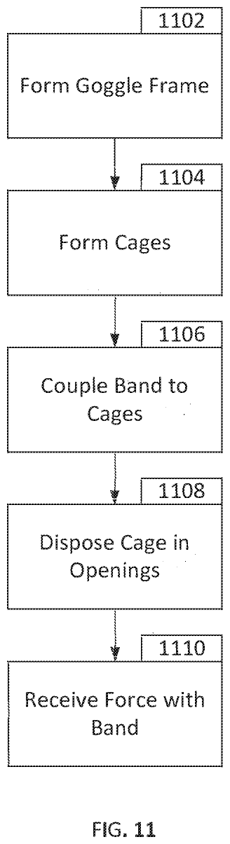

FIG. 11 shows a flowchart detailing a method of making and using the goggle of FIG. 1, in accordance with an embodiment of the invention. In block 1102, the goggle frame may be formed, as described herein. In certain embodiments, the goggle frame and the outriggers may be formed separately and the outriggers may be accordingly coupled to the goggle frame.

In block 1104, the cages may be formed, as described herein. The elastic band may be coupled to the cages in block 1106 by, for example, inserting the elastic band into the opening of the cages. In certain embodiments, for example, the elastic band may include a first end and a second end and the first end may be inserted into a first cage and the second end may be inserted into the second cage. Additionally, each cage may be composed of a plurality of portions. As such, the elastic band may be disposed within the plurality of portions and the portions may then be coupled together to form the cage and hold the end of the elastic band within the cage.

In block 1108, the cages may be disposed within the openings of the goggle frame and/or the outriggers, as described herein, to complete the goggle. The goggle may then be used in block 1110. In block 1110, a force may be received by the elastic band. The elastic band may stretch and exert a force on the coupling between the elastic band and the cage. Features of the cage (e.g., the protrusions) may hold the elastic band within the cage. The force may then be transferred to the opening through the cage. The cage may be held within the opening (e.g., through features of the cage such as the detents). As such, despite a force being exerted on the elastic band, the elastic band may remain coupled to the goggle frame through the cage.

It is appreciated that the embodiments described herein are for exemplary purposes only. Thus, various embodiments may include some or all of the features described herein. Furthermore, the steps described in FIGS. 10 and/or 11 may, in other embodiments, be performed in an order different from the order described in FIGS. 10 and/or 11.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A goggle comprising:
a goggle frame comprising a first end and a second end;
a first outrigger disposed on the first end of the goggle frame;
a second outrigger disposed on the second end of the goggle frame, wherein each of the first and second outriggers comprises an outrigger opening;
an elastic band comprising a first end and a second end;
a first cage disposed within the first outrigger opening; and
a second cage disposed within the second outrigger opening, wherein each of the first and second cages comprises a band opening and at least one protrusion disposed within the band opening, wherein the band opening of the first cage receives the first end of the elastic band, wherein the band opening of the second cage receive the second end of the elastic band, and wherein the at least one protrusion of each of the first cage and the second cage are coupled to the elastic band to hold the elastic band within the respective band opening.

2. The goggle of claim 1, wherein each of the first cage and the second cage further comprises a first cage fastener opening, wherein the elastic band further comprises a first band fastener opening, and wherein the goggle further comprises a first fastener configured to be disposed through both the first cage fastener opening and the first band fastener opening.

3. The goggle of claim 2, wherein each of the first cage and the second cage further comprises a second cage fastener opening, wherein the elastic band further comprises a second band fastener opening, wherein the goggle further comprises a second fastener configured to be disposed through both the second cage fastener opening and the second band fastener opening, and wherein the first cage fastener opening and the second cage fastener opening are disposed on opposing sides of the cage.

4. The goggle of claim 1, wherein each of the first cage and the second cage further comprises a detent, and wherein each of the first outrigger and the second outrigger further comprises a depression disposed within each of the respective outrigger opening and receiving the detent to hold the respective cage within the respective outrigger opening.

5. The goggle of claim 1, further comprising:
a goggle lens disposed within the goggle frame, wherein the first cage and/or the second cage is a metal cage, wherein at least one of the protrusions is a substantially triangular shaped teeth, wherein the elastic band opening comprises a substantially rectangular cross section, and wherein at least one of the protrusions extends into the substantially rectangular cross section.

6. A method of manufacturing the goggle of claim 1, the method comprising:
forming the goggle frame;
forming the first cage;
forming the second cage;
disposing the first cage within the first outrigger opening; and
disposing the second cage within the second outrigger opening.

7. The method of claim 6, wherein the first outrigger and the second outrigger are formed with the goggle frame.

8. The method of claim 6, further comprising:
forming the first outrigger;
forming the second outrigger; and
coupling the first outrigger and the second outrigger to the goggle frame.

9. The method of claim 6, wherein the forming the first cage comprises disposing the first end of the elastic band within the band opening of the first cage, and wherein the forming the second cage comprises disposing the second end of the elastic band within the band opening of the second cage.

10. A method of using the goggle of claim 1, the method comprising:
receiving a force on the elastic band;
stretching the elastic band due to the force; and
holding the first end and the second end of the elastic band within the respective band openings.

11. A cage configured to be disposed within a portion of a goggle frame, the cage comprising:
a cage body configured to be disposed within an opening of a goggle frame;
a band opening disposed within the cage body and configured to receive an elastic band; and
a protrusion disposed within the band opening and configured to couple to the elastic band to hold the elastic band within the band opening.

12. The cage of claim 11, wherein the cage further comprises a first cage fastener opening configured to receive a first fastener.

13. The cage of claim 12, wherein the cage further comprises a second cage fastener opening configured to receive a second fastener, and wherein the first cage fastener opening and the second cage fastener opening are disposed on opposing sides of the cage.

14. The cage of claim 12, wherein the cage body comprises a detent configured to be disposed within a depression of the goggle frame.

15. The cage of claim 12, wherein the cage body is metal, wherein the protrusion is a substantially triangular shaped tooth, wherein the band opening comprises a substantially rectangular cross section, and wherein the protrusion protrudes into the substantially rectangular cross section.

16. The cage of claim 12, wherein the cage body comprises a first portion and a second portion coupled to the first portion.

17. A method of manufacturing the cage of claim 11, the method comprising:
forming the cage body; and
forming the protrusion.

18. The method of claim 17, wherein the cage body and the protrusion are formed by machining a metal stock.

19. The method of claim 17, wherein the forming the cage body comprises forming the cage body from sheetmetal to define the band opening, and wherein the forming the protrusion comprises bending the protrusion into the band opening.

20. The method of claim 17, wherein the cage body and the protrusion are formed by three-dimensional printing.
